(19) United States
(12) Patent Application Publication
(10) Pub. No.: US 2017/0153225 A1
(43) Pub. Date: Jun. 1, 2017

(54) A METHOD OF SCREENING FOR A COMPOUND CAPABLE OF STIMULATING GLUCOSE TRANSPORT INTO BROWN AND/OR BRITE ADIPOCYTES OF A MAMMAL, A KIT FOR USE IN SUCH A METHOD

(71) Applicant: ATROGI AB, Stockholm (SE)

(72) Inventor: Tore Bengtsson, Vaxholm (SE)

(21) Appl. No.: 15/324,580

(22) PCT Filed: Jul. 9, 2014
(86) PCT No.: PCT/EP2014/064775

§ 371 Date: Jan. 6, 2017

(51) Int. Cl. G01N 33/50; G01N 33/68

(52) U.S. Cl. CPC G01N 33/5035; G01N 33/6872; G01N 33/5044; G01N 2800/042

(57) ABSTRACT

A method of screening for a candidate compound for use in the treatment of a condition involving dysregulation of metabolism in a mammal by identifying a compound capable of stimulating de novo synthesis of GLUT1 (Glucose transporter 1) in brown and/or brite adipocytes of the mammal and/or capable of stimulating translocation of GLUT1 in brown and/or brite adipocytes of the mammal. A kit for use in the method.

### FIELD OF THE INVENTION

The present invention relates to a screening method, in particular to a method of screening for a compound for the treatment of a condition involving a dysregulation of metabolism in a mammal, such as a dysregulation of energy homeostasis, glucose homeostasis or glucose uptake, as well as to a kit for use in such a method. In particular, the invention relates to a method of screening for a compound capable of stimulating glucose transport into brown and/or brite adipocytes of a mammal and to a kit for use in such a method for the treatment of a condition involving a dysregulation of metabolism in a mammal.

The invention also relates to a compound or combination of compounds for use in such treatment, a pharmaceutical composition comprising the compound or combination of compounds, and a method of treatment of such a condition by administration of such a compound or combination of compounds.

### BACKGROUND OF THE INVENTION

Brown adipose tissue (BAT) and brite (also referred to as beige) adipose tissue express the uncoupling protein 1 (UCP1) that is the primary site for thermogenesis and this tissue/these tissues can consume, in addition to free fatty acids, a very high amount of glucose from the blood that can both acutely and chronically affect energy utilization and glucose homeostasis in the mammal body (Nedergaard, Bengtsson et al. 2011, Cannon, Nedergaard 2004, Nedergaard, Bengtsson et al. 2010). Increasing energy utilization via increased fatty acid and glucose consumption in BAT and brite fat would thus lead to beneficial effects on metabolic diseases such as obesity and diabetes. Obesity affects a very large number of people in the world and obesity often leads to secondary diseases such as cardiovascular disease and diabetes. Diabetes comprises two distinct diseases, viz. type 1 (or insulin-dependent diabetes) and type 2 (insulin-independent diabetes), both of which involve the malfunction of glucose homeostasis. Type 2 diabetes affects more than 350 million people in the world and the number is rising rapidly. Complications of diabetes include severe cardiovascular problems, kidney failure, peripheral neuropathy, blindness and even loss of limbs and death in the later stages of the disease. Type 2 diabetes is characterized by insulin resistance in skeletal muscle and adipose tissue (fat), and at present there is no definitive treatment. Most treatments used today are focused on treating dysfunctional insulin signaling or inhibiting glucose output from the liver and many of those treatments have several drawbacks and side effects. There is thus a great interest in identifying novel insulin-independent ways to treat different form of metabolic disorders connected with obesity and dysregulation of glucose uptake such as type 2 diabetes.

In type 2 diabetes the insulin-signaling pathway is blunted in peripheral tissues such as fat and skeletal muscle. Methods for treating type 2 diabetes typically include lifestyle changes, as well as the administration of insulin or oral medications to help the body with the glucose homeostasis. People with type 2 diabetes in the later stages of the disease develop “beta-cell failure” or the inability of the pancreas to release insulin in response to high blood glucose levels. In the later stages of the disease patients often require insulin injections, in combination with oral medications, to manage their diabetes. In type 2 diabetes, the insulin-signaling pathway is blunted in peripheral tissues and most common drugs have side effects including the said down regulation or desensitization of the insulin pathway and/or the promotion of fat incorporation in fat, liver and skeletal muscle, furthermore increased stimulation of proliferation of certain cells and a higher risk of promoting cancer. There is thus a great interest in identifying novel ways to treat metabolic diseases including type 2 diabetes that do not include these side-effects. Activation of glucose uptake in BAT and brite adipose tissue would clear glucose from the blood and burn it in thermogenesis and would treat metabolic diseases including obesity and type 2 diabetes.

Insulin and catecholamines are released in the body in response to quite different stimuli. Whereas insulin is released in response to the rise in blood sugar levels after a meal, epinephrine (also referred to as adrenaline) and norepinephrine (also referred to as noradrenaline) are released due to various internal and external stimuli, such as exercise, emotions, stress and cold but also homeostatic tissue regulation. Insulin is an anabolic hormone that stimulates many processes involved in growth, including glucose uptake, glycogen and triglyceride formation whereas catecholamines are mainly catabolic. Although insulin and catecholamines normally have antagonistic effects, it has been shown previously that they have similar actions in skeletal muscle and brown fat on glucose uptake (Nevzorova, Evans et al. 2006, Cannon, Nedergaard 2004). Catecholamines stimulate glucose uptake via adrenergic receptors to supply muscle cells, brown fat and beige fat with an energy substrate.

Thus, it is likely that in mammals, including humans, the adrenergic and insulin systems can work independently to provide for the energy need of skeletal muscle and BAT and brite fat during different situations. Since insulin stimulates many anabolic processes including a number of unwanted side effects it would be beneficial to be able to stimulate glucose uptake in brown fat and brite fat.

It is known in the field of the art that adrenergic receptors are prototypic models for the study of G protein-coupled receptors (GPCRs) and their signaling (Santulli, Iaccarino 2013, Drake, Shenoy et al. 2006). There are three different classes of ARs, with distinct expression patterns and pharmacological profiles: α₁-, α₂- and β-ARs. The α₁-ARs comprise the α_{1A}, α_{1B} and α_{1D} while α₂-ARs are divided into α_{2A}, α_{2B} and α_{2C}. The β-ARs are also divided into the subtypes β₁, β₂, and β₃, of which β₂-AR is the major isoform in skeletal muscle cells (Watson-Wright, Wilkinson 1986, Liggett, Shah et al. 1988). Adrenergic receptors are G protein coupled and signal through second messengers such as cAMP and phospholipase C and are thus suited as prototypical models for most classes of GPCRs.

Glucose uptake in cells is mainly considered to be through facilitative glucose transporters (GLUT). GLUTs are transporter proteins mediating transport of glucose and/or fructose over the plasma membrane down the concentration gradient. There are fourteen known members of the GLUT family, named GLUT1-14, divided into three classes (Class I, Class II and Class III) dependent on their substrate specificity and tissue expression. GLUT1 and GLUT4 are the most intensively studied iso forms and, together with GLUT2 and GLUT3, belong to Class I which mainly transports glucose (in contrast to Class II that also transports fructose). GLUT1 is ubiquitously expressed and is responsible for basal glucose transport. GLUT4 is only expressed in peripheral tissues such as skeletal muscle, cardiac muscle and adipose tissues. GLUT4 has also been reported to be expressed in e.g. brain, kidney, and liver. GLUT4 is the major iso form involved in insulin stimulated glucose uptake.

To treat a condition involving a dysregulation of glucose homeostasis or glucose uptake in a mammal, it would be very advantageous to be able to activate glucose uptake through certain GLUTs. Regulation of GLUT1 translocation or intrinsic activity has been suggested to occur in several tissues including erythrocytes depending on ATP-levels (Hebert, Carruthers 1986). It has also been indicated in HEK-cells (Palmada, Boehmer et al. 2006), 3T3-L1 (Harrison, Clancy et al. 1992) and clone-9 cells (Barnes, Ingram et al. 2002). Impaired GLUT translocation, of in particular GLUT8, has been reported as involved in both male and female infertility (Gawlik, Schmidt et al. 2008, Carayannopoulos, Chi et al. 2000). The mechanism whereby insulin signaling increases glucose uptake is mainly via GLUT4-translocation from intracellular storage to the plasma membrane (Rodnick, Piper et al. 1992). After longer insulin stimulation also GLUT1-content is increased due to increased transcription (Taha, Mitsumoto et al. 1995). Glucose uptake in type 2 diabetes is associated with defects in PI3K activity, insulin receptor tyrosine, IRS and Akt phosphorylation, resulting in impairment of GLUT4 translocation to the plasma membrane. Impaired GLUT translocation also plays a role in muscle wasting. Furthermore, GLUT translocation plays a role in feeding behavior. Mice lacking GLUT4 develop problems with lipid and glucose homeostasis leading to changes in feeding behavior. Decreased concentrations of GLUT1 and GLUT3 have also been shown in the brains of patients with Alzheimer's disease (Simpson, Dwyer et al. 2008). Also in a review article of Shah K, et al. (Shah, Desilva et al. 2012) the role of glucose transporters in brain disease, diabetes and Alzheimer's disease is discussed.

The recent interest in research focusing on BAT and brites tissues that can express UCP1 stems from the insight that these tissues, when activated, expend energy in the form of heat production, thermogenesis, that can affect whole body energy homeostasis in humans, with recent evidence demonstrating the presence and function of BAT in adult humans (Nedergaard, Bengtsson et al. 2007). Besides its role in thermogenesis (Cannon, Nedergaard 2004), another important function is that BAT can consume, in addition to free fatty acids, a very high amount of glucose per gram tissue from the blood (Shibata, Perusse et al. 1989, Liu, Perusse et al. 1994). Studies in rodents have shown that the amount of glucose delivered to BAT is enough to both acutely and in long term affect glucose homeostasis (Stanford, Middelbeek et al. 2013). Because of these properties, BAT and brite fat may prove to be a potential therapeutic target for a number of metabolic disorders dependent on glucose homeostasis including obesity and type 2 diabetes.

Glucose uptake in BAT is stimulated in two metabolic states: sympathetically stimulated during active thermogenesis or by insulin during active anabolic processes. While insulin-stimulated glucose uptake in tissues, including BAT, is well-characterized by the phosphoinositide 3-kinase-phosphoinositide-dependent kinase-1-Akt (PI3K-PDK1-Akt) pathway resulting in the rapid translocation of glucose transporter 4 (GLUT4) from intracellular vesicles to the cell membrane (Huang, Czech 2007), the sympathetic pathway is poorly understood. Stimulation of the sympathetic nervous system via adrenoceptors which are prototypical G-protein-coupled receptors (GPCRs), predominately the β-adrenoceptor of the β₃-adrenoceptor subtype, increases non-shivering thermogenesis in mammal (Nedergaard, Bengtsson et al. 2007), but also increases glucose uptake in BAT (Inokuma, Ogura-Okamatsu et al. 2005). β₃-adrenoceptor stimulated glucose uptake is independent of the action of insulin in vivo and in vitro: glucose uptake in BAT in vivo is associated with decreases in plasma insulin levels (Shimizu, Saito 1991), while in vitro β-adrenoceptor mediated glucose uptake occurs in the absence of insulin (Marette, Bukowiecki 1989, Chernogubova, Cannon et al. 2004, Chemogubova, Hutchinson et al. 2005) and via actions at GLUT1 and not GLUT4 (Shimizu, Saito 1991, Dallner, Chemogubova et al. 2006). While other signaling pathways such as AMP-activated protein kinase can increase glucose uptake via an insulin-independent mechanism, it has been previously demonstrated that this mechanism is not likely to be involved in 33-adrenoceptor mediated glucose uptake in BAT (Hutchinson, Bengtsson 2005). Hence it has been unknown how GPCRs stimulate glucose uptake into UCP1 expressing cells. An alternative signaling pathway must be involved. One such candidate is mechanistic target of rapamycin (mTOR) (Laplante, Sabatini 2012).

mTOR is essential in control of many aspects of cell growth, metabolism and energy homeostasis. mTOR is the catalytic part of two functionally distinct multi-protein complexes: the well-studied mTOR complex 1 (mTORC1) and the less studied mTOR complex 2 (mTORC2). They have different downstream targets, biological functions and importantly, different sensitivity to the drug rapamycin. mTORC1 is pharmacologically inhibited by short-term rapamycin treatment, whereas mTORC2 is resistant to short-term rapamycin treatment, although long-term treatment prevents mTORC2 complex assembly (Phung, Ziv et al. 2006). Recent studies of mTOR show that both complexes have important regulatory roles in white adipose tissue (Lamming, Sabatini 2013). Most of the efforts have however been put in studying white adipose tissue leaving the role and the importance of both complexes of mTOR in BAT function relatively unexplored. Recent data indicates a role of mTORC2 in glucose homeostasis, with adipose-specific ablation of rictor, a component of the mTORC2 complex, depressing insulin-stimulated glucose uptake in adipose tissue and impairing glucose tolerance in vivo (Kumar, Lawrence et al. 2010). Adipose specific deletion of raptor, component of mTORC1 complex, however results in mice resistant to diet-induced obesity and is insulin sensitive (Polak, Cybulski et al. 2008), indicating vastly different roles for mTORC1 and mTORC2 in adipose tissues.

### SUMMARY OF THE INVENTION

It has been found that mTOR is necessary for GPCR stimulated glucose uptake in mammalian brown adipocytes. Stimulation of GPCRs increases glucose uptake solely via a pathway divided into two parts that both are fully necessary for glucose uptake. The first part (A) involves de novo synthesis of GLUT1; this part is not dependent on either of the mTOR complexes. The second part (B) involves translocation of GLUT1 to the plasma membrane by an mTORC2 mediated pathway. In the art it has been believed (Inokuma, Ogura-Okamatsu et al. 2005) that activation of UCP1 function in BAT and brite fat tissue itself would lead to increased glucose uptake. However our surprising finding shows that the glucose uptake mechanism can and must be stimulated separately from the UCP1 function.

Both parts (A) and (B) are fully necessary in order for the glucose uptake mechanism to function optimally in brown and brite fat cells, and the presence of this mechanism proves surprisingly that this process is not coupled to UCP1 function. Compounds that should stimulate energy uptake in UCP1 expressing cells, such as brown and brite adipocytes, must thus stimulate both part (A) and part (B) to activate glucose uptake and for BAT and brite fat tissue to reach full physiological effect.

Thus, it has been surprisingly found that non-insulin dependent glucose uptake in adipocytes expressing UCP1 is not dependent on UCP1 but fully dependent on a pathway divided into two parts. The first part (A) of the pathway involves de novo synthesis of GLUT1 and is independent of mTORC2 activity. The second part (B) of the pathway involves translocation of GLUT1 to the plasma membrane and is dependent of mTORC2 activity. Furthermore, it has been surprisingly found that the non-insulin dependent stimulated glucose uptake is not coupled with UCP1 function. However, it is known that glucose uptake is necessary for the thermogenesis that is the major function of BAT and brite fat tissue (Nedergaard, Bengtsson et al. 2011, Cannon, Nedergaard 2004, Nedergaard, Bengtsson et al. 2010). In view of this, the above-mentioned novel and surprising finding shows that a compound or combination of compounds, capable of stimulating both (A) GLUT1 de novo synthesis and (B) translocation of GLUT1 to the plasma membrane of a cell, would provide a means to treat metabolic diseases such as obesity and type 2 diabetes by stimulation of thermogenesis in BAT and brite fat

Drugs provided by the present invention, or drug candidate compounds identified by the screening method of the present invention, that stimulate glucose uptake in BAT and/or brite fat, by stimulating de novo synthesis of GLUT1 in brown and/or brite fat cells and translocation of GLUT1 in said cells, can be used to treat metabolic disorders, in particular related to dysregulation of glucose transport including insulin resistance or hyperglycemia, type 2 diabetes, inadequate glucose tolerance, obesity, polycystic ovary syndrome (PCOS), hypertension and the metabolic syndrome.

A major problem with obesity and type 2 diabetes is that peripheral tissues become insulin resistant and glucose uptake is blunted. According to the present invention, this can be treated with a compound or a combination of compounds that stimulates parts (A) and (B) and thereby upregulates glucose uptake in brown and/or brite adipocytes. Upregulating glucose uptake via a compound or a combination of compounds that stimulates parts (A) and (B) reduces requirement of insulin or insulin mimetic drugs. Accordingly, the incidence of life threating complications of obesity and type 2 diabetes can be reduced. Such approach could also be therapeutically useful in other human diseases that are induced by, regulated by, or associated with, changes in glucose homeostasis.

In one aspect the invention is directed to a compound capable of stimulating de novo synthesis of GLUT1 in brown and/or brite adipocytes and translocation of GLUT1 in brown and/or brite adipocytes for use in the treatment of a metabolic disorder as defined herein, in particular related to dysregulation of glucose transport including insulin resistance or hyperglycemia, type 2 diabetes, inadequate glucose tolerance, obesity, polycystic ovary syndrome (PCOS), hypertension and the metabolic syndrome.

Therefore, in one aspect there is provided a method of screening for a candidate compound for use in the treatment of a condition involving dysregulation of metabolism in a mammal, said method comprising identifying a compound capable of stimulating de novo synthesis of GLUT1 in brown and/or brite adipocytes and of stimulating translocation of GLUT1 in brown and/or brite adipocytes.

In another aspect, the invention is directed to a combination comprising:

(a) a compound capable of stimulating de novo synthesis of GLUT1 in brown and/or brite adipocytes; and

(b) a compound capable of stimulating translocation of GLUT1 in brown and/or brite adipocytes, for use in the treatment of a metabolic disorder as defined herein, in particular related to dysregulation of glucose transport, including insulin resistance or hyperglycemia, type 2 diabetes, inadequate glucose tolerance, obesity, polycystic ovary syndrome (PCOS), hypertension and the metabolic syndrome; and to a method for identifying either a compound (a), or a compound (b) or both a compound (a) and a compound (b), for use in such a combination.

The combination of compounds (a) and (b) may be a physical combination, where the compounds are included in one and the same pharmaceutical composition. However, it should be realized that the combination of compounds (a) and (b) is not limited to such physical combination. For example, compounds (a) and (b) may be separately formulated and used together in a combination therapy, whereby they may be administered together or separately, at different time points, as is well-known to the person of ordinary skill in the art.

Therefore, in one aspect there is provided a method of screening for a candidate compound for use in combination with a compound capable of stimulating GLUT1 translocation in brown and/or brite adipocytes for the treatment of a condition involving dysregulation of metabolism in a mammal, said method comprising identifying a compound capable of stimulating de novo synthesis of GLUT1 in brown and/or brite adipocytes.

Likewise, in one aspect there is provided a method of screening for a candidate compound for use in combination with a compound capable of stimulating de novo synthesis of GLUT1 in brown and/or brite adipocytes, for the treatment of a condition involving dysregulation of metabolism in a mammal, said method comprising identifying a compound capable of stimulating GLUT1 translocation in brown and/or brite adipocytes.

In some embodiments, a method of screening for a candidate compound for use in the treatment of a condition involving dysregulation of metabolism in a mammal comprises:
bringing a compound into contact with at least one population of cells, comprising cells that are capable of synthesizing GLUT1,
determining de novo synthesis of GLUT1 in said population of cells,
bringing the compound into contact with at least one population of cells, comprising cells that are capable of translocating GLUT1,
determining translocation of GLUT1 in said population of cells, and
identifying the candidate compound based on the determined de novo synthesis of GLUT1 and the determined translocation of GLUT1.

In some embodiments, a method of screening for a candidate compound for use in combination with a compound capable of stimulating GLUT1 translocation in brown and/or brite adipocytes, for the treatment of a condition involving dysregulation of metabolism in a mammal, comprises:
bringing a compound into contact with at least one population of cells, comprising cells that are capable of synthesizing GLUT1,
determining de novo synthesis of GLUT1 in said population of cells,
identifying the candidate compound based on the determined de novo synthesis of GLUT1.

In some embodiments, a method of screening for a candidate compound for use in combination with a compound capable of stimulating de novo synthesis of GLUT1 in adipocytes for the treatment of a condition involving dysregulation of metabolism in a mammal, comprises:
bringing a compound into contact with at least one population of cells, comprising cells that are capable of translocating GLUT1,
determining translocation of GLUT1 in said population of cells,
identifying the candidate compound based on the determined translocation of GLUT1.

The population of cells that may be used in a screening method of the invention may comprise any cells capable of de novo synthesis of GLUT1 and/or of translocating GLUT1 from cell interior to cell plasma membrane, but preferably comprises mammalian cells, more preferably selected from adipocytes, such as brown fat cells, brite fat cells and white fat cells; in particular brown fat cells and brite fat cells.

Another aspect is a kit for use in a method of the invention, said kit comprising cells capable of de novo synthesis of GLUT1 and/or of translocating GLUT1, together with instructions for use of the kit.

Cells provided in the kit of the present invention may comprise any cells capable of de novo synthesis of GLUT1 and/or of translocating GLUT1 from cell interior to cell plasma membrane, but preferably comprises mammalian cells, more preferably selected from adipocytes, such as brown fat cells, brite fat cells and white fat cells; in particular brown fat cells and brite fat cells, or cells that may be considered representative for such cells.

One further aspect is a compound for use in a method of treatment or prevention of a condition involving dysregulation of metabolism in a mammal, by administering, to a mammal in need of such treatment or prevention, a therapeutically effective amount of a compound which stimulates de novo synthesis of GLUT1 and translocation of GLUT1 in brown and/or brite adipocytes of the mammal.

Still a further aspect is method of treatment or prevention of a condition involving dysregulation of metabolism in a mammal, by administering, to a mammal in need of such treatment or prevention, a therapeutically effective amount of a compound which stimulates (A) de novo synthesis of GLUT1 and (B) translocation of GLUT1 in brown and/or brite adipocytes of the mammal.

### BRIEF DESCRIPTION OF THE DRAWINGS

FIG. 1 is a schematic representation of the mechanism of glucose uptake in brown and brite adipocytes, showing the two parts (A) and (B) necessary for the uptake, i.e. the GLUT1 de novo synthesis (A) which does not depend on mTORC2, and the GLUT1 translocation (B), which does depend on mTORC2.

FIG. 2. GPCR stimulation of glucose uptake in mature brown adipocytes primary cultures. Isoproterenol 1 μM which is a stimulator of the prototypical GPCRs β-adrenergic receptors increase glucose uptake (p=0.0042) in brown mature brown adipocytes in culture. Inhibition of isoproterenol stimulated glucose uptake with Torin-1 (1 μM) which is a specific mTOR inhibitor leads to full inhibition of glucose uptake. Each value represents the mean±S.E.M. (n=4-6).

FIG. 3. GPCR stimulation of glucose uptake in brown fat in mice. Isoproterenol stimulated glucose uptake in vivo in FVB mice. Mice were injected with 1 mg/kg ip isoproterenol. Isoproterenol stimulated glucose uptake was significantly reduced (p=0.0175) by the mTOR inhibitor KU 0063794 (1 μM). Each value represents the mean±S.E.M. (n=5).

FIG. 4. GPCR stimulation of total GLUT1 content in mature brown adipocytes. Western blot of GLUT1 protein content in mature brown adipocytes was quantified after isoproterenol stimulation (1 μM, 0-2 h) in presence or absence of 1 μM KU 0063794. Isoproterenol significantly increase the amount of GLUT1 in a mTOR independent manner (p=0.0067 and p=0.0017 respectively) (n=3).

FIG. 5. GPCR stimulated de novo synthesis of GLUT1 is not inhibited by mTOR inhibition. Permeabilized mature brown adipocytes treated 2 h with 1 μM isoproterenol (p=0.0334) showing total cellular GLUT1 in the presence or absence of 1 μM KU 0063794. (n=3). The figure shows that de novo synthesis can be stimulated and that this process is not inhibited by inhibition of mTOR.

FIG. 6. GPCR stimulated translocation of GLUT1 to the plasma membrane is inhibited by mTOR inhibition. mTOR inhibitor KU 0063794 prevents the translocation of GLUT1 to the plasma membrane after stimulation of isoproterenol in mature brown adipocytes. Non-permeabilized mature brown adipocytes after stimulation of isoproterenol for 2 hours and inhibition of KU. Histogram shows significant inhibition of GLUT1 transport to the plasma membrane (n=3). *(p<0.05)

FIG. 7. GPCR stimulated glucose uptake in primary brown adipocytes are blocked by inhibition of mTORC2. Knock down of rictor with K2 transfection system resulted in a reduction of rictor protein and inhibition of isoproterenol stimulated glucose uptake (p=0.0105). Each value represents the mean±S.E.M (n=3).

FIG. 8. mTORC2 is a key factor in isoproterenol mediated glucose uptake and can be used a marker for GPCR stimulated translocation of GLUT1. mTOR phosphorylation on Ser2481 but not on Ser2448 in response to isoprenaline (1 μM) in the presence or absence of mTOR inhibitor KU 0063794 (1 μM) by western blot. The immunoblot is representative of three experiments performed. Each value represents the mean±S.E.M (n=2). ***(p<0.001).

### DETAILED DESCRIPTION OF THE INVENTION

By a condition involving a dysregulation of metabolism in a mammal, e.g. a condition involving a dysregulation of glucose homeostasis or glucose uptake, is meant a condition such as insulin resistance or hyperglycemia, type 2 diabetes, inadequate glucose tolerance, obesity, polycystic ovary syndrome (PCOS), hypertension and the metabolic syndrome.

It should be realized that even when identified as a “candidate compound” as defined herein, the compound may have to pass various other tests, e.g. pharmacological, clinical and toxicological tests and so on, before being able to be used as a drug. Thus, the expression “candidate compound” for use as defined herein, e.g. in for use in the treatment of a condition involving dysregulation of metabolism in a mammal, should not be generally construed as a statement that the method permits to positively identify a compound for use in such a treatment, but rather should be understood as an indication that the method permits to identify a compound that may have a usefulness in such treatment.

The word “cell” when used herein, generally refers to a population of cells, and not to one single cell, unless the contrary is specified or apparent from the context. Any cell used in the present invention preferably is a mammalian cell, e.g. a mammalian adipocyte, such as a brown adipocyte (i.e. brown fat cell) or a brite (also referred to as “beige”) adipocyte (brite fat cell).

A population of mammalian cells for use in the present invention comprises cells that have been separated from a mammalian body, or that originates from cells separated from a mammalian body, and that kept in culture using method well-known to the person of ordinary skill in the field.

By GLUT1 is meant the protein Glucose transporter 1, also referred to as solute carrier family 2, or facilitated glucose transporter member 1 (SLC2A1).

The term “de novo synthesis” of a complex molecule generally refers to the biochemical pathway where a complex biomolecule is synthesized anew from simple molecules. By de novo synthesis of GLUT1 thus is meant the biochemical process of transcription and synthesis of functional GLUT1 protein in a cell.

By “stimulating” etc. is meant the action of causing, either directly or indirectly, an increase or enhancement of some process or activity (which thus is “stimulated”).

By “translocation” of GLUT1 is meant the “migration” of GLUT1 from the interior of the cell to the cell membrane.

The terms “fat cell” and “adipocyte” herein are used as interchangeable synonyms.

The inventive method of screening for a candidate compound for use in the treatment of a condition involving dysregulation of metabolism in a mammal comprises identifying a compound capable of stimulating (A) de novo synthesis of GLUT1 (Glucose transporter 1) in brown and/or brite adipocytes of the mammal and capable of stimulating (B) translocation of GLUT1 in brown and/or brite adipocytes of the mammal.

In some embodiments, the method comprises:

(i) bringing a compound into contact with at least one population of cells, comprising cells that are capable of synthesizing GLUT1,

(ii) determining de novo synthesis of GLUT1 in said population of cells,

(iii) bringing the compound into contact with at least one population of cells, comprising cells that are capable of translocating GLUT1,

(iv) determining translocation of GLUT1 in said population of cells, and

(v) identifying the candidate compound based on the determined de novo synthesis of GLUT1 and the determined translocation of GLUT1.

In some embodiments of a screening method according to the invention, the cells are grown in a cell culture medium, transferred into a sample well of a conventional microplate having e.g. 8, 12, 24, 48, 96, 384 or 1536 sample wells, cell differentiation is induced by addition of a differentiation medium, and the cells are allowed to differentiate for a suitable time period. The cells are then brought into contact with the compound for a predetermined time period, of e.g. 5 minutes to 10 hours, or 0.5 hour to 5 hours, e.g. 1 hour to 3 hours.

A suitable cell for use in the screening method of the invention may be derived e.g. from primary cultures from cells that can express UCP1 at some point such as brown fat, white fat, brite/beige fat but can also comprise cells that do not express UCP1 such as cells from heart, skeletal muscle, liver, brain, mammal gland and other mammalian tissues. The cell or cells to be used in the screening method generally is selected so as to be representative of the fatty tissue(s) involved. The cell suitably is selected from brown fat or brite fat cells or cells representative of brown fat or brite fat cells, in particular cells that can express UCP1 at some point (i.e. during the life of the cell). In some embodiments, the cells used in the screening method of the invention are brown fat cells.

Examples of cell lines that can be utilized include fat cell lines, such as hMADS, HIB cells, 3T3-L1, 3T3 F442 and heart cell lines such as H9c2, VH 2, skeletal muscle cell lines, such as L6, L8, C2C12 and other cell lines, well known to the person of ordinary skill in the art.

Cell lines of different origin with GPCRs and/or GLUT1 can also be used. Although a number of cell types can be used for this process, one that can be transfected and express (or overexpress) GPCRs and/or GLUT1 would be preferable, for example CHO cells. The introduced GPCR and/or GLUT1 could be stably transfected or non-stably transfected according to methods well known to investigators of skill in the art.

The compound generally is provided dissolved in a liquid phase, which e.g. may be an aqueous phase, such as purified water or a suitably buffered and isotonic aqueous phase, or an organic solvent phase, or a mixture thereof. The compound is brought into contact with the cells at a concentration that suitably should correspond to an amount relevant for pharmaceutical use, e.g. a concentration of 10⁻⁸ to 10⁻¹ M, or 10⁻⁷ to 10⁻² M, e.g. 10⁻⁶ to 10⁻³ M.

De novo synthesis of GLUT1 can be measured as increased amount of GLUT1 protein in a cell after stimulation. This can be for example be measured with Western blot from total cell lysate or from fraction of cells or any other method measuring GLUT1 content in the cell known in the art. De novo synthesis can also be measured with immunohistochemistry and other methods dependent on antibodies against GLUT1 such a flow cytometry. De novo synthesis can also be predicted by measuring gene expression of GLUT1 in a cell known in the art.

The GLUT1 translocation may be determined by measuring any parameter P_{GLUT1} which is a measurable parameter that may be considered representative for the translocation of a GLUT1 in the cell. For example, P_{GLUT} may be the GLUT1 translocation as measured by use of a method as described in (Koshy et al. 2010).

In some embodiments, GLUT1 translocation is determined by measuring the content of GLUT1 in the plasma membrane.

As noted herein, activation of mTORC2 results in enhanced GLUT1 translocation in a cell expressing GLUT1. Therefore the activity of mTORC2 also may be determined as representative for GLUT1 translocation in a cell expressing GLUT1.

In some embodiments, the method therefore comprises:
bringing the compound into contact with a population of cells, e.g. adipocytes, such as brown or brite adipocytes, that express mTOR, said cells being capable of activating mTORC2, and
determining the activity of mTORC2 in said cells as a measure of GLUT1 translocation in said cells.

The activity of mTORC2 may be determined e.g. by measuring the kinase activity of mTORC2, e.g. using an in vitro assay as in (Huang J., 2012) in Methods Mol Biol. 2012; 821:75-86.

Since mTORC2 is activated by phosphorylation of mTOR, the activity of mTORC2 also may be determined by measuring phosphorylation of mTOR, in particular phosphorylation of S2448 and/or S2481 of mTOR, especially phosphorylation of S2481.

Thus, in some embodiments, the activity of mTORC2 determined in a cell brought into contact with a compound (mTORC2_{comp}) is compared to the activity of mTORC2 (mTORC2_{ref}), determined for a similar cell which has not been brought into contact with the compound, such as a cell treated with buffer only under similar conditions. In such case, for the compound be contemplated as a candidate compound according to the invention, mTORC2_{comp} should be higher than mTORC2_{ref}.

The reference value also may be the mTORC2 activity obtained when bringing cells expressing mTOR into contact with a compound having a determined or previously known mTORC2 activating capacity, such as isoproterenol. Thus, in some embodiments, the activity of mTORC2 determined for cells brought into contact with a compound to be screened (mTORC2_{comp}) is compared to the mTORC2 activity (mTORC2_{agonist}), determined for similar cells brought into contact with a compound of a known mTORC2 activating effect, such as isoproterenol.

In some embodiments the method of screening for a candidate compound for the treatment of a condition involving dysregulation of metabolism in a mammal, further comprises:
bringing a compound into contact with at least one population of cells, comprising cells that express mTOR and GLUT1, e.g. brown and/or brite adipocytes; and
determining hexose uptake, e.g. glucose uptake, in cells brought into contact with the compound.

It is known that mTORC2 may be activated in response to a signal from a GPCR. Therefore, in some embodiments, the cells that express mTOR also express a GPCR.

The candidate compound is identified based on the determined de novo synthesis of GLUT and translocation of GLUT and/or mTORC2 activity in cells brought into contact with the compound to be screened. Preferably, the screening method involves comparing the determined values of the de novo synthesis of GLUT1 and translocation of GLUT1, with reference values.

For example, the determined values representative for GLUT1 synthesis or GLUT1 translocation may be compared with corresponding values determined in similar population(s) of cells under similar conditions, but which cells have not been brought into contact with the compound. The identification also may comprise comparing values representative for GLUT1 synthesis or GLUT1 translocation determined for cells that have been brought into contact with different concentrations of the compound.

According to one aspect, the invention provides a method for identifying GPCR ligands that stimulate GLUT1 translocation to the plasma membrane and glucose uptake, and which therefore will provide for a treatment for a condition involving a dysregulation of glucose homeostasis or glucose uptake in a mammal.

Adrenergic receptors are considered prototypical for GPCRs and have been investigated extensively (Santulli, Iaccarino 2013, Drake, Shenoy et al. 2006). In some embodiments, therefore, the GPCR is an adrenergic receptor (AR). In some particular embodiments, the GPCR is an alpha-AR. In some embodiments, the GPCR is an α₁-AR. In some other embodiments, the GPCR is an α₂-AR. In still other embodiments, the GPCR is a β-AR. In some embodiments, the GPCR is a β₁-AR. In other embodiments, the GPCR is β₂-AR. In other embodiments, the GPCR is a β₃-AR

In some embodiments, the screening method may include a preliminary screening of substances to identify compounds that bind to GPCRs, i.e. compounds that are GPCR ligands. Such preliminary identification of ligands for GPCRs may be accomplished using e.g. in silico methods or methods using preparations of plasma membrane from tissue. In such a preliminary screening, a cell free assay system based on protein-protein interaction can also be used, such as one using electrochemiluminiscence.

Thus, by use of cell-free methods, compounds that bind GPCRs can be identified in a preliminary screening step. Preferable molecules identified in such a method are small molecules with a molecular weight less than or equal to 1000 Daltons. These compounds are then screened in the cell-based screening method as described herein.

The screening method according to the present invention is not limited to any particular compounds, i.e. the compound may be any pharmaceutically acceptable substance, e.g. a known pharmaceutical substance.

In some embodiments, compounds that are previously known GPCR ligands can be screened in the method of the invention, in order to identify such GPCR ligands that cause an increase in de novo synthesis of GLUT1 and/or an increase in mTORC2 activity and GLUT1 translocation.

A preferable compound for screening in the method of the invention is one that may be administered orally in order to enhance glucose uptake in brown and brite fat tissue.

In some advantageous embodiments, a candidate compound identified according to the present invention is one that causes an increase in glucose uptake in brown and/or brite fat cells, but that does not cause an increase in glucose uptake in white fat cells.

In some embodiments, the candidate compound is identified based on the determined de novo synthesis of GLUT1 and translocation of GLUT1 and/or mTORC2 activity in brown and/or brite adipocytes brought into contact with the compound to be screened. For example, the screening method may involve comparing de novo synthesis of GLUT1 and translocation of GLUT1 and/or mTORC2 activity determined in cells brought into contact with the compound, with reference values.

The reference value for the mTORC2 activity e.g. may be the mTORC2 activity obtained when bringing cells expressing mTOR into contact with a compound having a determined or previously known mTORC2 activating capacity, such as isoproterenol. Thus, in some embodiments, the activity of mTORC2 determined for cells brought into contact with a compound to be screened (mTORC2_{comp}) is compared to the mTORC2 activity (mTORC2_{agonist}), determined for similar cells brought into contact with a compound of a known mTORC2 activating effect, such as isoproterenol.

The screening method may suitably be performed using a brown or brite adipocyte as target cell type, or a cell representative for a brown or brite adipocyte. The screening method may also be expanded to a panel comprising any number of different cells, thereby allowing for the verification of a selectivity of the compound for the target cell type.

Thus, in some embodiments, the screening method is performed using cells representative for brown and/or brite fat cells, as well as cells representative for white fat cells. In such embodiments, the screening method of the invention may allow to identify a compound having selective stimulating effect on glucose uptake in brown and/or brite fat cells, over white fat cells.

A screening method of the invention may involve the use of a panel of cells representative also for other types of tissues, e.g. muscle cells, beta cells, brain cells, liver cells, reproductive cells and cells involved in reproduction, and mammary cells.

In some embodiments of the screening method of the invention, at least the first cell comprises a GPCR. In some embodiments, both cells comprise a GPCR.

By the screening method of the present invention, compounds may be identified for the treatment of a condition involving a dysregulation of metabolism in a mammal, in particular a condition involving a dysregulation of glucose homeostasis or glucose uptake in the mammal.

In one aspect, thus, there is provided a method of screening for a candidate compound, for use in the treatment of a condition involving a dysregulation of glucose homeostasis or glucose uptake in a mammal, comprising identifying a compound that stimulates (i.e. causes an increase of) GLUT1 de novo synthesis and/or GLUT1 translocation in brown and/or brite fat cells of the mammal.

In another aspect, there is provided a method of screening for a candidate compound (a) for use in combination with a compound (b) capable of stimulating GLUT1 translocation in adipocytes, for the treatment of a condition involving dysregulation of metabolism in a mammal, comprising identifying a compound (a) that causes an increase of GLUT1 de novo synthesis in brown and/or brite fat cells of the mammal.

In some embodiments, said screening method comprises steps (i) and (ii) as described herein above, whereby the candidate compound (a) is identified based on the determined de novo synthesis GLUT1.

The compound (b) capable of stimulating GLUT1 translocation, with which the identified candidate compound (a) is used, may be one identified by a method as also described herein. However, it should be realized that the compound (b) capable of stimulating GLUT1 translocation may also be one identified by any other method, or one previously known as stimulating GLUT1 translocation.

In another aspect, there is provided a method of screening for a candidate compound (b) for use in combination with a compound (a) capable of stimulating de novo synthesis of GLUT1 in adipocytes, for the treatment of a condition involving dysregulation of metabolism in a mammal, comprising identifying a compound (b) that stimulates GLUT1 translocation in brown and/or brite fat cells of the mammal.

In some embodiments, said screening method comprises steps (iii) and (iv) as described herein above, whereby the candidate compound is identified based on the determined translocation of GLUT1.

The compound (a) capable of stimulating GLUT1 de novo synthesis, with which the identified candidate compound (b) is used, may be one identified by a method as also described herein. However, it should be realized that the compound (a) capable of stimulating GLUT1 de novo synthesis may also be one identified by any other method, or one previously known as stimulating GLUT1 de novo synthesis.

In some embodiments, the method of screening for a candidate compound for use in the treatment of a condition involving dysregulation of metabolism in a mammal comprises identifying a compound or combination of compounds capable of stimulating (A) de novo synthesis of GLUT1 (Glucose transporter 1) in brown adipocytes of the mammal and capable of stimulating (B) translocation of GLUT1 in brown adipocytes of the mammal.

In some embodiments, the method of screening for a candidate compound for use in the treatment of a condition involving dysregulation of metabolism in a mammal comprises identifying a compound or combination of compounds capable of stimulating (A) de novo synthesis of GLUT1 (Glucose transporter 1) in brown adipocytes of the mammal and capable of stimulating (B) translocation of GLUT1 in brown adipocytes of the mammal.

It should be realized that the expressions “determining de novo synthesis of GLUT1” and “determining the translocation of GLUT1”, as used herein, do not necessarily mean providing an absolute quantitative measure of parts (A) and (B), as defined herein above, and also does not necessarily imply that a quantitative measure of any change in (A) and/or (B) must be provided. It will be clear to the person of ordinary skill that what is necessary is to ascertain whether bringing the compound in contact with the cells of the population causes any positive effect at all on (A) and/or (B).

In some embodiments, the screening method of the invention also comprises administering the compound or combination of compounds to a test animal, e.g. a laboratory rodent, and determining glucose uptake in brown and/or brite fat of the animal, and optionally also in other tissues of the body, e.g. white fat.

In one aspect a kit is provided, for use in a method of screening for a candidate compound for the treatment of a condition involving a dysregulation of metabolism in a mammal, e.g. a dysregulation of glucose homeostasis or glucose uptake, said kit comprising cells capable of de novo synthesis of GLUT i, and cells capable of translocation of GLUT i, together with instructions for use of the kit.

In some embodiments, the kit comprises a cell capable of expressing a GPCR and of expressing mTOR.

In some embodiments, the kit comprises a cell capable of expressing a GPCR and of expressing a GLUT.

In some embodiments, the kit comprises a compound that is a known mTORC2 agonist, such as isoproterenol, for use as a reference in the determination of mTORC2 activity.

In some embodiments, the kit comprises a GPCR agonist, such as noradrenaline.

A cell for use in a kit of the invention e.g. is derived from primary cultures from heart, skeletal muscle, brown fat, white fat, brite/beige fat, liver, brain, mammal gland and other mammalian tissues. The cell or cells to be used in the kit generally is selected so as to be representative of the tissue(s) involved or afflicted by the condition, disease or disorder. For example, if the kit is for use in a screening method directed to identifying a compound useful in the treatment of a neurodegenerative disorder, the cell suitably is selected from mammalian nerve cells or cells representative of mammalian nerve cells or cells that may have an importance in the functioning of the mammalian nervous system, in particular in the transportation of glucose into the mammalian nervous system, e.g. into the brain. Likewise, if the kit is for use in a screening method directed to identifying a compound useful in the treatment of a metabolic disorder, such as diabetes, the cell suitably is selected from mammalian muscle cells or cells representative of mammalian muscle cells, in particular mammalian skeletal muscle cells.

Examples of cell lines that can be used in the kit of the present invention include heart cell lines such as H9c2, VH 2, skeletal muscle cell lines, such as L6, L8, C2C12, fat cell lines, such as HIB cells, 3T3-L1, 3T3 F442 and other cell lines, well known to the person of ordinary skill in the art.

Cell lines of different origin with introduced mTOR and/or and/or GPCR and/or GLUT can also be included in the kit of the invention, e.g. a cell that is transfected and expresses (or overexpresses) any of said proteins, for example a CHO cell line.

Depending on the cellular context, any of the mentioned activities will lead to alteration and/or increase in the GPCR signaling cascade coupled to glucose uptake, resulting in improvements relevant to the disease states of interest as will be discussed in detail herein below.

Thus, in one embodiment of the invention, there is provided a method of treatment of a mammal subject, preferably a human, suffering from or susceptible to develop a disease that is induced by, regulated by, or associated with, changes in glucose homeostasis, by a compound that upregulates translocation of GLUT1, in brown fat and/or brite fat of said subject.

In another aspect an increase of de novo synthesis of GLUT1 and GLUT1 translocation in brown fat will lead to increased glucose uptake from the blood to prevent diabetes and related disorders.

In another aspect an increase of GLUT1 de novo synthesis and GLUT1 translocation in brown adipose tissue or brite/beige adipose tissues will lead to increased glucose uptake from the blood into the tissue, which may be useful in treatment of a condition involving a dysregulation of glucose metabolism in a mammal, such as diabetes. Therefore, in one embodiment of the invention, a method is provided, for the treatment of diabetes by administration of a compound capable of stimulating de novo synthesis of GLUT1 and translocation of GLUT1.

One aspect of the present invention relates to a method of treatment of a condition involving a dysregulation of glucose homeostasis or glucose uptake in a mammal, comprising the administration of a therapeutic effective amount of one or more compounds that bind GPCR, said binding causing an increase of mTORC2 activity and, thereby, GLUT1 translocation in brown and/or brite fat cells of the mammal, to a mammal in need of such treatment.

Another aspect of the present invention relates to the use of a compound identified in a screening method of the present invention, in the manufacturing of a medicament for use in the treatment of a condition involving a dysregulation of glucose homeostasis or glucose uptake in a mammal.

Still another aspect relates to a pharmaceutical composition comprising a compound identified in a screening method of the present invention. Still another aspect relates to a compound identified in a screening method of the present invention.

Therapeutically effective means an amount of compound, or of a combination of compounds, which is effective in producing GLUT1 de novo synthesis and GLUT1 translocation in brown and/or brite fat cells of a mammal. Administration means delivering the compound of the present invention to a mammal by any method for example, orally, intravenously, intramuscularly, topically, transdermal, or inhalation.

Carriers for the administration include any carrier known in the art including water, saline, phosphate buffered saline, dextrose, glycerol, ethanol and similar carriers and combination of these. Carriers can also comprise wetting or emulsifiers, preservatives or buffers that enhance effectiveness, half-life, and shelf life of the compound(s).

Furthermore additional carriers influencing the release of the compound(s) including how quick, sustained or delayed the active compound(s) is released when administered to the mammal.

The composition of this invention can be any form including solid, semi-solid and liquid such as used in tablets, pills, powders, solutions, dispersions, suspensions, liposomes suppositories, injections and infusible solutions.

The methods and compositions of the invention can be administered to any suitable mammal such as rabbit, rat or mouse or more preferable a human.

While this invention has been described with respect to various specific examples it is to be understood that the invention is not limited by this and it can be variously practiced within the scope of the claims. The invention is further illustrated by the following non-limiting examples.

### EXAMPLES

### Example 1

Brown fat primary cells were isolated from NMRI mice (3-4 week-old) purchased from Nova-SCB AB, Sweden. All experiments were conducted with ethical permission (N388/12) from the North Stockholm Animal Ethics Committee. Animals were euthanized by CO₂, and brown fat precursor cells were isolated from the intrascapular, axillary and cervical brown adipocyte depots as previously described (Hutchinson, Bengtsson 2006, Hutchinson, Chernogubova et al. 2006). The tissue was minced and transferred to a HEPES-buffered solution (pH 7.4) containing 0.2% (wt/vol) crude collagenase type II. Routinely, tissue from 6 mice was digested in 10 ml of the HEPES-buffered solution. The tissue was digested for 30 minutes at 37° C., with constant vortexing. The digest was filtered through a 250 μm filter and the solution incubated on ice for 15 min to allow the mature adipocytes and fat droplets to float. The infranatant was filtered through a 25 μm filter, centrifuged (10 min, 700×g), the pellet resuspended in DMEM (4.5 g D-glucose/l) and recentrifuged. The pellet was finally resuspended in 0.5 ml cell culture medium per mouse dissected. The cell culture medium consisted of DMEM (4.5 g D-glucose/l) supplemented with 10% newborn calf serum, 2.4 nM insulin, 10 nM HEPES, 50 IU/ml penicillin, 50 μg/ml streptomycin and 25 μg/ml sodium ascorbate. Aliquots of 0.1 ml cell suspension were cultured in 12-well culture dishes with 0.9 ml of cell culture medium. Cultures were incubated in a 37° C. humidified atmosphere of 8% CO₂ in air. On days 1, 3 and 5, the cell culture medium was renewed. Cells were used on day 7.

Glucose uptake in vitro was performed as previously described (Yamamoto, Hutchinson et al. 2007). On day 7 the cells were treated with inhibitors for 30 min before addition of isoproterenol for 2 hours, unless otherwise indicated. 10 min before 2-deoxy-D-[1-³H]-glucose ([³H]-2DG) uptake measurement, the medium was discarded, and cells were washed with prewarmed PBS (10 mM phosphate buffer, 2.7 mM KCl, 137 mM NaCl, pH 7.4). Glucose-free DMEM (containing 0.5% BSA, 0.25 mM sodium ascorbate) was added and drugs were re-added with trace amounts of 2-deoxy-D-[1-³H]-glucose (50 nM) (specific activity 7.5 Ci/mmol) for 10 min. Reactions were terminated by washing in ice-cold PBS, cells lysed (400 μl of 0.2 M NaOH, 1 h at 60° C.) and the incorporated radioactivity determined by liquid scintillation counting.

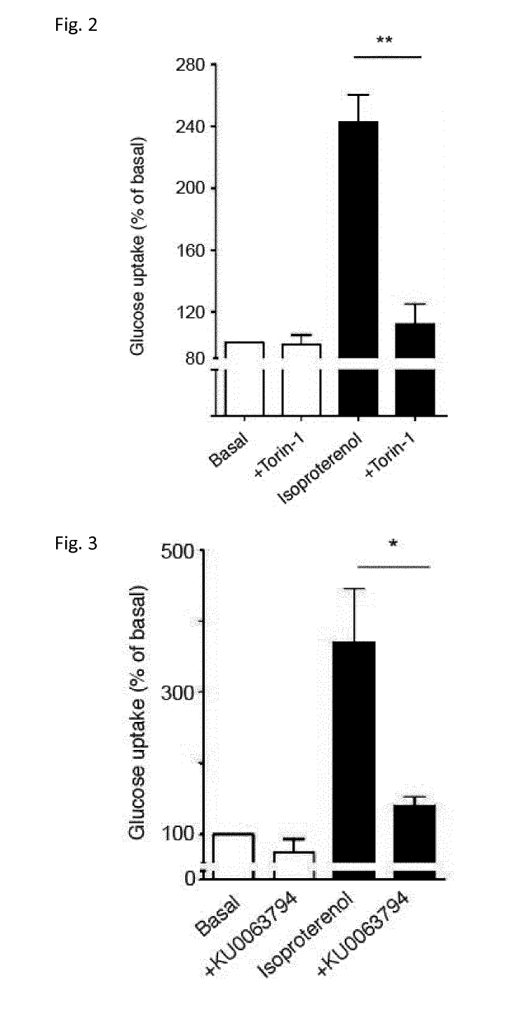

Example 1 shows that GPCRs stimulation, such as 3-adrenergic stimulation via isoproterenol, increases glucose uptake in mature primary brown adipocytes (FIG. 2).

It has been previously shown that mature primary brown adipocytes do not express UCP1 in significant levels if not previously challenged with norepinephrine (Nedergaard, Petrovic et al. 2005). Example 1 thus also shows that glucose uptake in mature primary brown adipocytes is not dependent on UCP1 function. Example 1 further shows that cells that do not express UCP1, but that at some point can express UCP1, such as primary brown adipocytes and brite adipocytes, can be used for screening for a candidate compound to increase glucose uptake in such cells. Furthermore, Example 1 shows that inhibition of mTOR fully inhibits 3-adrenergic stimulated glucose uptake, showing that glucose uptake in such cells is fully dependent on a mechanism not dependent on UCP1, but dependent on mTOR.

### Example 2

Groups of FVB mice were fasted for 5 hours prior to study and anesthetized with pentobarbital (60 mg per kg of body weight, i.p.). Mice were then injected with a specific mTOR inhibitor KU 0063794 (10 mg/kg i.p.) or DMSO as control. Isoproterenol (1 mg/kg ip) or saline were injected after 10 min and [³H]-2DG (130 μCi/kg body weight ip) 20 min prior to end time. BAT was dissected 1 hour after [³H]-2DG injection, and tissues digested with 0.5M NaOH overnight. Glucose uptake was measured by liquid scintillation counting. All experiments were conducted with ethical permission (N388/12) from the North Stockholm Animal Ethics Committee. Increased glucose uptake in BAT has previously been shown to treat dysregulation of metabolisms in mammals (Nedergaard, Bengtsson, et al. 2011; Cannon, Nedergaard 2004; Nedergaard, Bengtsson, et al. 2010). Example 2 thus shows that GPCR can increase glucose uptake in BAT in mammals and that this mechanism is fully through a mechanism dependent on mTOR and not UCP1 (FIG. 3). Cells that do not express UCP1, but at some point can express UCP1, can thus be used for screening for a candidate compound for use in the treatment of a condition involving dysregulation of metabolism in a mammal.

### Example 3

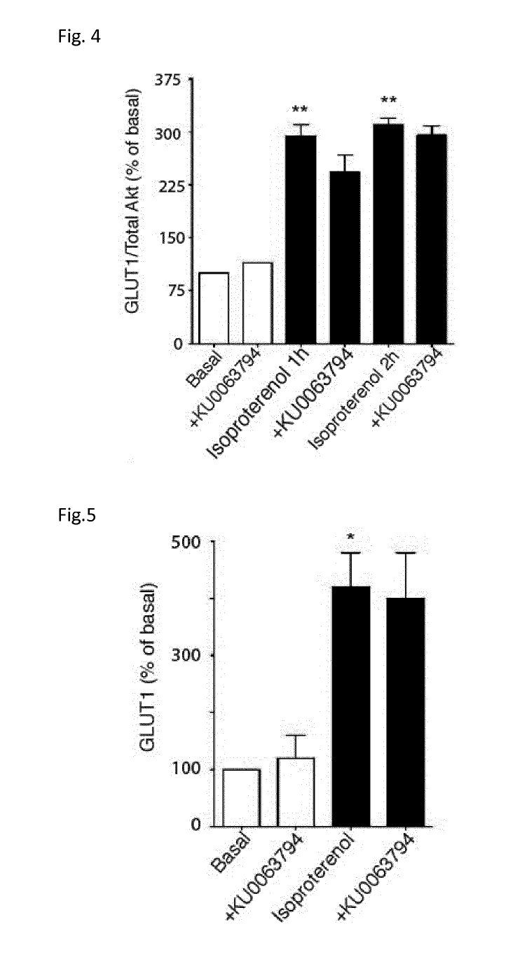

Brown adipocytes were grown as described in Example 1 and differentiated in 12 well plates and serum starved the night prior to experiment. On day 7 the cells were challenged with inhibitors for 30 min before being stimulated with drugs as indicated. Lysates were prepared in prewarmed (65° C.) sample buffer (62.5 mM Tris pH 6.8, 2% SDS, 10% glycerol, 50 mM dithiothreitol, 0.1% bromophenol blue) and boiled for 5 min. Samples were loaded on a 8 or 12% acrylamide gel and separated for 2 hours at 100 V. Proteins were transferred to Hybond-P polyvinylidene difluoride membranes (pore size 0.45 m; Amersham Biosciences, Arlington Heights, Ill.). The primary GLUT1 antibody (diluted 1:1000) was from AbCam (Cambridge, UK). The primary antibody was detected using a secondary antibody (horseradish peroxidase-linked anti-rabbit IgG, Cell Signaling) diluted 1:2000 and enhanced chemiluminescence (ECL, Amersham Biosciences). Images were quantified using Image J 1.46r. GPCR stimulation with isoproterenol lead to increased total GLUT1 content in mature brown adipocytes (FIG. 4). Western blot of GLUT1 protein content in mature brown adipocytes was quantified after isoproterenol stimulation (1 μM, 0-2 h) in presence or absence of 1 μM KU 0063794. Isoproterenol significantly increase the amount of GLUT1 in an mTOR independent manner (p=0.0067 and p=0.0017 respectively) (n=3).

Example 3 shows that GPCR stimulation increase de novo synthesis of GLUT1 and that this mechanism is not dependent on mTOR.

### Example 4

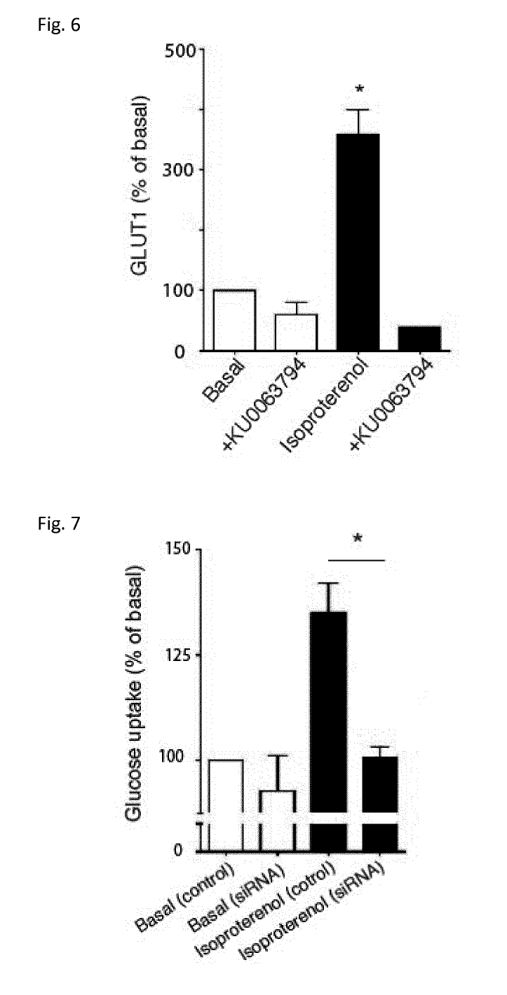

Brown adipocytes were isolated as described in Example 1 and seeded onto BD Falcon culture chamber slides (BD Biosciences, Franklin Lakes, N.J.). Cells were serum starved the night prior to experiment. On day 7 the cells were challenged with inhibitors for 30 min before being stimulated for 1-2 hours with drugs as indicated. Cells were washed with warm PBS and fixed for 15 min (4% formaldehyde in PBS). Cells were washed with PBS and formaldehyde quenched with 50 mM glycine in PBS, and washed three times for 5 min each with PBS. Cells were blocked for 1 h at room temperature with 8% BSA in PBS, and washed three times for 5 min each with PBS. For permeabilizing the cells, the cells were treated with 10% triton x in PBS (dilution 1:40) before blocking. Primary antibody (2 μg/ml GLUT1 antibody (AbCam, Cambridge, UK), 1.5% BSA in PBS) was added and slides incubated over night at 4° C. The next day the cells were washed three times for 5 min each with PBS. Slides were then incubated with secondary antibody (3 μg/ml alexa488 conjugated goat antirabbit IgG (Invitrogen, Paisley, UK), 3% BSA in PBS) and washed three times for 5 min each with PBS. Slides were mounted with mounting media (8% 1,4-diazabicyclooctane, 75% glycerol in PBS) and sealed. Images was observed in an inverted laser-scanning microscope (LSM 510META; Carl Zeiss, Advances Imaging Microscopy, Jena, Germany). FIG. 5 shows the total cellular GLUT1 content (% of basal) in permeabilized mature brown adipocytes treated 2 h with 1 μM isoproterenol (p=0.0334), in the presence or absence of 1 μM KU 0063794. (n=3), while FIG. 6 shows GLUT1 content of the plasma membrane (% of basal) in non-permeabilized mature brown adipocytes following 2 h of 1 μM isoproterenol treatment in the presence or absence of 1 μM KU 0063794 (p=0.0153).

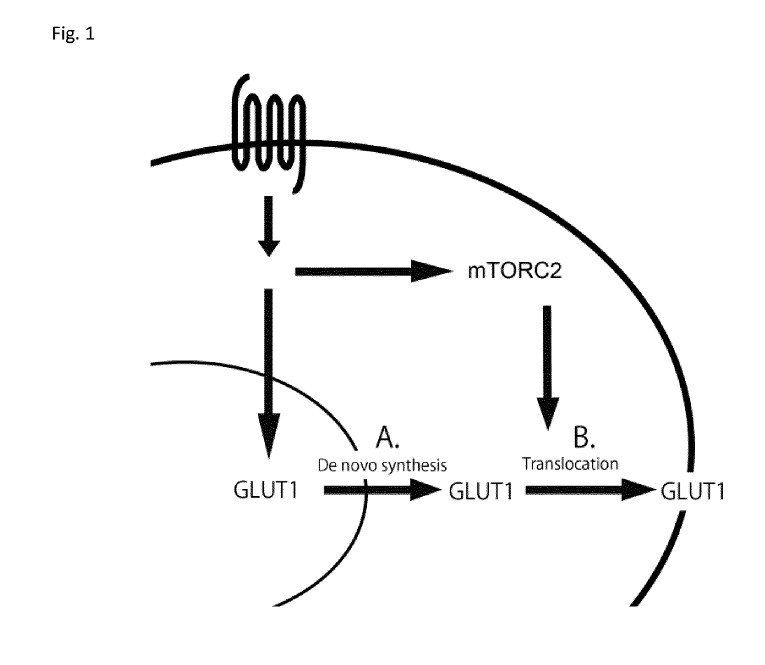

Example 4 thus shows that the stimulated de novo synthesis is uncoupled from UCP1 function and not dependent on active mTORC2 (FIG. 5), as also schematically illustrated in FIG. 1. Example 4 also shows that inhibition of mTORC2 causes significant inhibition of GLUT1 translocation to the plasma membrane (FIG. 6). Example 4 further shows that GPCR can stimulate translocation of GLUT1 to the plasma membrane and that the novo synthesis of GLUT1 and the translocation of GLUT1 are not coupled to each other, but occur through separate mechanisms. Example 4 further shows that the same compound can stimulate both de novo synthesis and translocation of GLUT1 in cells that do not express UCP1, but that at some point can express UCP1.

Example 4 also may be seen as an illustration of a screening method of the invention, where the compound isoproterenol represents a candidate compound.

### Example 5

Primary brown adipocytes were used on day 3 and transfected with K2 Transfection System (Biontex laboratories) according to manufacturer's protocol using 2.5 μg/ml of siRNA (sequence: CAGAAAGCAATCGCAACTCACCACA) directed against mouse rictor (from Sigma). 24 h after transfection, glucose uptake or western blotting was performed as described above. The results are illustrated in FIG. 7 and show that GPCR stimulated glucose uptake in primary brown adipocytes are fully inhibited by knockdown of mTORC2. mTOR is the catalytic part of two functionally distinct multi-protein complexes: the well-studied mTOR complex 1 (mTORC1) and the mTOR complex 2 (mTORC2). Since the entire glucose uptake is inhibited by knockdown of mTORC2 the example also shows that mTORC2 is the complex involved in translocation of GLUT1 to the plasma membrane as exemplified in FIG. 6, and as schematically illustrated in FIG. 1. Example 5 further shows that GPCR stimulated glucose uptake is dependent on translocation to the plasma membrane by specific mechanism dependent on mTORC2.

### Example 6

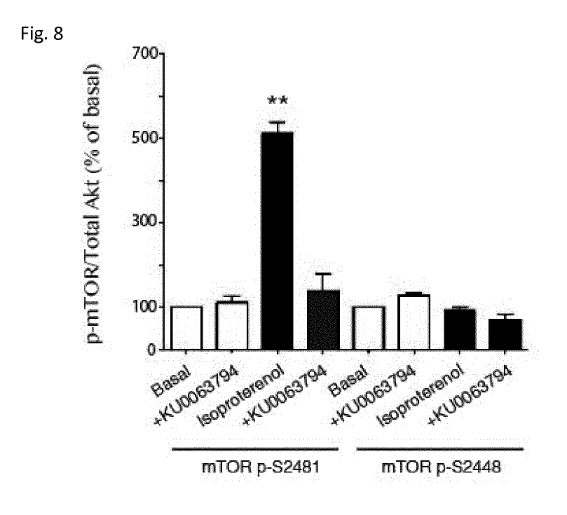

Phosphorylation of mTOR was measured using antibodies for phosphorylation at Ser2481 and phosphorylation at Ser2448 of mTOR. Phosphorylation at Ser2481 (p=0.0042), but not at Ser2448, in response to 2 hours stimulation by 1 μM isoprenaline in the presence or absence of mTOR inhibitor KU 0063794 (1 μM) is shown in FIG. 8. The immunoblot is representative of three experiments performed. Example 6 shows that mTOR phosphorylation can be used as a measurement for GPCR activation of mTOR. Example 6 also shows that phosphorylation at Ser2481 on mTOR can be used as a measurement for translocation of GLUT1 to the plasma membrane. Example 6 together with FIGS. 6 and 7 further show that phosphorylation at Ser2481 on mTOR is indicative of translocation of GLUT1 and increased glucose uptake.

### REFERENCE LIST

BARNES, K., INGRAM, J. C., PORRAS, O. H., BARROS, L. F., HUDSON, E. R., FRYER, L. G., FOUFELLE, F., CARLING, D., HARDIE, D. G. and BALDWIN, S. A., 2002. Activation of GLUT1 by metabolic and osmotic stress: potential involvement of AMP-activated protein kinase (AMPK). Journal of cell science, 115(Pt 11), pp. 2433-2442.
CANNON, B. and NEDERGAARD, J., 2004. Brown adipose tissue: function and physiological significance. Physiological Reviews, 84(1), pp. 277-359.
CARAYANNOPOULOS, M. O., CHI, M. M., CUI, Y., PINGSTERHAUS, J. M., MCKNIGHT, R. A., MUECKLER, M., DEVASKAR, S. U. and MOLEY, K. H., 2000. GLUT8 is a glucose transporter responsible for insulin-stimulated glucose uptake in the blastocyst. Proceedings of the National Academy of Sciences of the United States of America, 97(13), pp. 7313-7318.
CHERNOGUBOVA, E., CANNON, B. and BENGTSSON, T., 2004. Norepinephrine increases glucose transport in brown adipocytes via beta3-adrenoceptors through a cAMP, PKA, and PI3-kinase-dependent pathway stimulating conventional and novel PKCs. Endocrinology, 145(1), pp. 269-280.
CHERNOGUBOVA, E., HUTCHINSON, D. S., NEDERGAARD, J. and BENGTSSON, T., 2005. Alpha1- and beta1-adrenoceptor signaling fully compensates for beta3-adrenoceptor deficiency in brown adipocyte norepinephrine-stimulated glucose uptake. Endocrinology, 146(5), pp. 2271-2284.
DALLNER, O. S., CHERNOGUBOVA, E., BROLINSON, K. A. and BENGTSSON, T., 2006. Beta3-adrenergic receptors stimulate glucose uptake in brown adipocytes by two mechanisms independently of glucose transporter 4 translocation. Endocrinology, 147(12), pp. 5730-5739.
DRAKE, M. T., SHENOY, S. K. and LEFKOWITZ, R. J., 2006. Trafficking of G protein-coupled receptors. Circulation research, 99(6), pp. 570-582.
GAWLIK, V., SCHMIDT, S., SCHEEPERS, A., WENNEMUTH, G., AUGUSTIN, R., AUMULLER, G., MOSER, M., AL-HASANI, H., KLUGE, R., JOOST, H. G. and SCHURMANN, A., 2008. Targeted disruption of Slc2a8 (GLUT8) reduces motility and mitochondrial potential of spermatozoa. Molecular membrane biology, 25(3), pp. 224-235.
HARRISON, S. A., CLANCY, B. M., PESSINO, A. and CZECH, M. P., 1992. Activation of cell surface glucose transporters measured by photoaffinity labeling of insulin-sensitive 3T3-L1 adipocytes. Journal of Biological Chemistry, 267(6), pp. 3783-3788.
HEBERT, D. N. and CARRUTHERS, A., 1986. Direct evidence for ATP modulation of sugar transport in human erythrocyte ghosts. The Journal of biological chemistry, 261(22), pp. 10093-10099.
HUANG, J., 2012 An in vitro assay for the kinase activity of mTOR complex 2 Methods Mol Biol. 821:75-86
HUANG, S. and CZECH, M. P., 2007. The GLUT4 glucose transporter. Cell metabolism, 5(4), pp. 237-252.
HUTCHINSON, D. S. and BENGTSSON, T., 2006. AMP-activated protein kinase activation by adrenoceptors in L6 skeletal muscle cells: mediation by alpha1-adrenoceptors causing glucose uptake. Diabetes, 55(3), pp. 682-690.
HUTCHINSON, D. S. and BENGTSSON, T., 2005. alpha1A-adrenoceptors activate glucose uptake in L6 muscle cells through a phospholipase C-, phosphatidylinositol-3 kinase-, and atypical protein kinase C-dependent pathway. Endocrinology, 146(2), pp. 901-912.
HUTCHINSON, D. S., CHERNOGUBOVA, E., SATO, M., SUMMERS, R. J. and BENGTSSON, T., 2006. Agonist effects of zinterol at the mouse and human beta(3)-adrenoceptor. Naunyn-Schmiedeberg's archives ofpharmacology, 373(2), pp. 158-168.
INOKUMA, K., OGURA-OKAMATSU, Y., TODA, C., KIMURA, K., YAMASHITA, H. and SAITO, M., 2005. Uncoupling protein 1 is necessary for norepinephrine-induced glucose utilization in brown adipose tissue. Diabetes, 54(5), pp. 1385-1391.
KOSHY, S., ALIZADEH, P., TIMCHENKO, L. T. & BEETON, C. 2010, Quantitative measurement of GLUT4 translocation to the plasma membrane by flow cytometry, Journal of visualized experiments: JoVE, vol. (45). pii: 2429. doi, no. 45, pp. 10.3791/2429.
KUMAR, A., LAWRENCE, J. C., Jr, JUNG, D. Y., KO, H. J., KELLER, S. R., KIM, J. K., MAGNUSON, M. A. and HARRIS, T. E., 2010. Fat cell-specific ablation of rictor in mice impairs insulin-regulated fat cell and whole-body glucose and lipid metabolism. Diabetes, 59(6), pp. 1397-1406.
LAMMING, D. W. and SABATINI, D. M., 2013. A Central role for mTOR in lipid homeostasis. Cell metabolism, 18(4), pp. 465-469.
LAPLANTE, M. and SABATINI, D. M., 2012. mTOR signaling in growth control and disease. Cell, 149(2), pp. 274-293.
LIGGETT, S. B., SHAH, S. D. and CRYER, P. E., 1988. Characterization of beta-adrenergic receptors of human skeletal muscle obtained by needle biopsy. The American Journal of Physiology, 254(6 Pt 1), pp. E795-8.
LIU, X., PERUSSE, F. and BUKOWIECKI, L. J., 1994. Chronic norepinephrine infusion stimulates glucose uptake in white and brown adipose tissues. The American Journal of Physiology, 266(3 Pt 2), pp. R914-20.
MARETTE, A. and BUKOWIECKI, L. J., 1989. Stimulation of glucose transport by insulin and norepinephrine in isolated rat brown adipocytes. The American Journal of Physiology, 257(4 Pt 1), pp. C714-21.
NEDERGAARD, J., BENGTSSON, T. and CANNON, B., 2011. New powers of brown fat: fighting the metabolic syndrome. Cell metabolism, 13(3), pp. 238-240.
NEDERGAARD, J., BENGTSSON, T. and CANNON, B., 2010. Three years with adult human brown adipose tissue. Annals of the New York Academy of Sciences, 1212, pp. E20-36.
NEDERGAARD, J., BENGTSSON, T. and CANNON, B., 2007. Unexpected evidence for active brown adipose tissue in adult humans. American journal of physiology. Endocrinology and metabolism, 293(2), pp. E444-52.
NEDERGAARD, J., PETROVIC, N., LINDGREN, E. M., JACOBSSON, A. and CANNON, B., 2005. PPARgamma in the control of brown adipocyte differentiation. Biochimica et biophysica acta, 1740(2), pp. 293-304.
NEVZOROVA, J., EVANS, B. A., BENGTSSON, T. and SUMMERS, R. J., 2006. Multiple signalling pathways involved in beta2-adrenoceptor-mediated glucose uptake in rat skeletal muscle cells. British journal of pharmacology, 147(4), pp. 446-454.
PALMADA, M., BOEHMER, C., AKEL, A., RAJAMANICKAM, J., JEYARAJ, S., KELLER, K. and LANG, F., 2006. SGK1 kinase upregulates GLUT1 activity and plasma membrane expression. Diabetes, 55(2), pp. 421-427.
PHUNG, T. L., ZIV, K., DABYDEEN, D., EYIAH-MENSAH, G., RIVEROS, M., PERRUZZI, C., SUN, J., MONAHAN-EARLEY, R. A., SHIOJIMA, I., NAGY, J. A., LIN, M. I., WALSH, K., DVORAK, A. M., BRISCOE, D. M., NEEMAN, M., SESSA, W. C., DVORAK, H. F. and BENJAMIN, L. E., 2006. Pathological angiogenesis is induced by sustained Akt signaling and inhibited by rapamycin. Cancer cell, 10(2), pp. 159-170.
POLAK, P., CYBULSKI, N., FEIGE, J. N., AUWERX, J., RUEGG, M. A. and HALL, M. N., 2008. Adipose-specific knockout of raptor results in lean mice with enhanced mitochondrial respiration. Cell metabolism, 8(5), pp. 399-410.
RODNICK, K. J., PIPER, R. C., SLOT, J. W. and JAMES, D. E., 1992. Interaction of insulin and exercise on glucose transport in muscle. Diabetes care, 15(11), pp. 1679-1689.
SANTULLI, G. and IACCARINO, G., 2013. Pinpointing beta adrenergic receptor in ageing pathophysiology: victim or executioner? Evidence from crime scenes. Immunity & ageing: I & A, 10(1), pp. 10-4933-10-10.
SHAH, K., DESILVA, S. and ABBRUSCATO, T., 2012. The Role of Glucose Transporters in Brain Disease: Diabetes and Alzheimer's Disease. International journal of molecular sciences, 13(10), pp. 12629-12655.
SHIBATA, H., PERUSSE, F., VALLERAND, A. and BUKOWIECKI, L. J., 1989. Cold exposure reverses inhibitory effects of fasting on peripheral glucose uptake in rats. The American Journal of Physiology, 257(1 Pt 2), pp. R96-101.
SHIMIZU, Y. and SAITO, M., 1991. Activation of brown adipose tissue thermogenesis in recovery from anesthetic hypothermia in rats. The American Journal of Physiology, 261(2 Pt 2), pp. R301-4.
SIMPSON, I. A., DWYER, D., MALIDE, D., MOLEY, K. H., TRAVIS, A. and VANNUCCI, S. J., 2008. The facilitative glucose transporter GLUT3: 20 years of distinction. American journal of physiology. Endocrinology and metabolism, 295(2), pp. E242-53.
STANFORD, K. I., MIDDELBEEK, R. J., TOWNSEND, K. L., AN, D., NYGAARD, E. B., HITCHCOX, K. M., MARKAN, K. R., NAKANO, K., HIRSHMAN, M. F., TSENG, Y. H. and GOODYEAR, L. J., 2013. Brown adipose tissue regulates glucose homeostasis and insulin sensitivity. The Journal of clinical investigation, 123(1), pp. 215-223.
TAHA, C., MITSUMOTO, Y., LIU, Z., SKOLNIK, E. Y. and KLIP, A., 1995. The insulin-dependent biosynthesis of GLUT1 and GLUT3 glucose transporters in L6 muscle cells is mediated by distinct pathways. Roles of p21ras and pp70 S6 kinase. The Journal of biological chemistry, 270(42), pp. 24678-24681.
WATSON-WRIGHT, W. M. and WILKINSON, M., 1986. The muscle slice—a new preparation for the characterization of beta-adrenergic binding in fast- and slow-twitch skeletal muscle. Muscle & nerve, 9(5), pp. 416-422.
YAMAMOTO, D. L., HUTCHINSON, D.S. and BENGTSSON, T., 2007. Beta(2)-Adrenergic activation increases glycogen synthesis in L6 skeletal muscle cells through a signalling pathway independent of cyclic AMP. Diabetologia, 50(1), pp. 158-167.

1. A method of screening for a candidate compound for use in the treatment of a condition involving dysregulation of metabolism in a mammal, said method comprising identifying a compound capable of stimulating de novo synthesis of GLUT1 (Glucose transporter 1) in brown and/or brite adipocytes of the mammal and capable of stimulating translocation of GLUT1 in brown and/or brite adipocytes of the mammal.

2. The method of claim 1, comprising:
bringing a compound into contact with a population of cells, comprising cells that are capable of synthesizing GLUT1,
determining de novo synthesis of GLUT1 in said population of cells,
bringing the compound into contact with a population of cells, comprising cells that are capable of translocating GLUT1,
determining translocation of GLUT1 in said population of cells, and
identifying the candidate compound based on the determined de novo synthesis of GLUT1 and the determined translocation of GLUT1.

3. A method of screening for a candidate compound for use in combination with a compound capable of stimulating GLUT1 translocation in brown and/or brite adipocytes of a mammal, for the treatment of a condition involving dysregulation of metabolism in the mammal, said method comprising identifying a compound capable of stimulating de novo synthesis of GLUT1 in brown and/or brite adipocytes of the mammal.

4. The method of claim 3, comprising:
bringing a compound into contact with a population of cells, comprising cells that are capable of synthesizing GLUT1,
determining de novo synthesis of GLUT1 in said population of cells,
identifying the candidate compound based on the determined de novo synthesis of GLUT1.

5. A method of screening for a candidate compound for use in combination with a compound capable of stimulating de novo synthesis of GLUT1 in brown and/or brite adipocytes of a mammal, for the treatment of a condition involving dysregulation of metabolism in the mammal, said method comprising identifying a compound capable of stimulating GLUT1 translocation in brown and/or brite adipocytes of the mammal.

6. The method of claim 5, comprising:
bringing a compound into contact with a population of cells, comprising cells that are capable of translocating GLUT1,
determining translocation of GLUT1 in said population of cells,
identifying the candidate compound based on the determined translocation of GLUT1.

7. The method of claim 2, wherein the candidate compound is identified by determining an increase in GLUT1 de novo synthesis compared to a reference value.

8. The method of claim 2, wherein the candidate compound is identified by determining an increase in GLUT1 translocation compared to a reference value.

9. The method of claim 1, wherein at least one population of cells comprises mammalian cells.

10. The method of claim 2, wherein at least one population of cells comprises adipocytes.

11. The method of claim 10, wherein at least one population of cells comprises brown adipocytes.

12. The method of claim 10, wherein at least one population of cells comprises brite adipocytes.

13. The method of claim 1, wherein the condition involving dysregulation of metabolism in a mammal is selected from insulin resistance or hyperglycemia, type 2 diabetes, inadequate glucose tolerance, obesity, polycystic ovary syndrome (PCOS), hypertension and the metabolic syndrome.

14. The method of claim 13, wherein the condition is type 2 diabetes.

15. A kit for use in a method according to claim 1, comprising cells capable of synthesizing GLUT1 and/or of translocating GLUT1, together with instructions for use thereof.

16. The method of claim 4, wherein the candidate compound is identified by determining an increase in GLUT1 de novo synthesis compared to a reference value.

17. The method of claim 6, wherein the candidate compound is identified by determining an increase in GLUT1 translocation compared to a reference value.

18. The method of claim 4, wherein at least one population of cells comprises adipocytes.

19. The method of claim 6, wherein at least one population of cells comprises adipocytes.
